(19) United States
(12) Patent Application Publication
(10) Pub. No.: US 2025/0050494 A1
(43) Pub. Date: Feb. 13, 2025

(54) SURGICAL ROBOTIC SYSTEMS AND INSTRUMENT DRIVE UNITS THEREOF

(71) Applicant: Covidien LP, Mansfield, MA (US)

(72) Inventors: Ranjan K. Mishra, Orange, CT (US); Michael A. Zemlok, Prospect, CT (US); Eric J. Taylor, Southington, CT (US)

(21) Appl. No.: 18/723,651

(22) PCT Filed: Jan. 25, 2023
(86) PCT No.: PCT/IB2023/050637

§ 371 Date: Jun. 24, 2024

Related U.S. Application Data
(60) Provisional application No. 63/303,543, filed on Jan. 27, 2022.

(51) Int. Cl. B25J 9/10; A61B 34/30; B25J 13/08; B25J 19/00

(52) U.S. Cl. CPC B25J 9/102; A61B 34/30; B25J 13/088; B25J 19/0041

(57) ABSTRACT

A surgical robotic system for use in a minimally invasive surgical procedure includes an instrument drive unit having five degrees of rotational freedom.

### CROSS-REFERENCE TO RELATED APPLICATIONS

The present application is a U.S. National Stage Application filed under 35 U.S.C. § 371(a) of International Application Serial No. PCT/IB2023/050637 filed on Jan. 25, 2023, which claims the benefit of provisional U.S. Patent Application No. 63/303,543 filed on Jan. 27, 2022.

### FIELD

The present technology is generally related to surgical robotic systems used in minimally invasive medical procedures.

### BACKGROUND

Some surgical robotic systems include a console supporting a surgical robotic arm and a surgical instrument or at least one end effector (e.g., forceps or a grasping tool) mounted to the robotic arm. The robotic arm provides mechanical power to the surgical instrument for its operation and movement. Each robotic arm may include an instrument drive unit operatively connected to the surgical instrument and coupled to the robotic arm via a rail. In operation, the robotic arm is moved to a position over a patient and then guides the surgical instrument into a small incision via a surgical trocar or a natural orifice of a patient to position the end effector at a work site within the patient's body. The instrument drive unit drives a rotation of each corresponding driven member of the attached surgical instrument to perform a surgical treatment.

### SUMMARY

In one aspect of the disclosure, a robotic arm assembly is provided and includes a robotic arm, and an instrument drive unit configured to provide five degrees of rotational freedom and coupled to the robotic arm. The instrument drive unit includes a casing configured to rotate about a longitudinal axis thereof, and a cluster of electric motor assemblies supported in the casing and rotatable with the casing. One of the electric motor assemblies includes a magnetic rotor rotatably supported in the casing, a motor stator coil fixed to and positioned within the casing, and an output shaft rotatably coupled to the magnetic rotor. The output shaft includes a proximal end portion non-rotatably coupled to the magnetic rotor, a distal end portion configured to interface with a corresponding driven member of a surgical instrument, and a plurality of flexure sections positioned between the proximal and distal end portions of the output shaft and configured to flex under a torque load. The instrument drive unit further includes a proximal encoder positioned proximally of the flexure sections and configured to determine a rotational position of the proximal end portion of the output shaft, and a distal encoder positioned distally of the flexure sections and configured to determine a rotational position of the distal end portion of the output shaft. A difference between the determined rotational positions corresponds with a torque of the output shaft.

In aspects, the instrument drive unit may further include an electronic disc assembly supported on the casing and including a plurality of printed circuit boards. Each of the printed circuit boards may be in electrical communication with a respective motor assembly of the plurality of motor assemblies.

In aspects, each of the motor assemblies may include a planetary gear set coupling the respective magnetic rotor with the respective output shaft.

In aspects, the robotic arm assembly may further include a base coupled to the robotic arm and a roll drive assembly. The base may rotatably support the instrument drive unit thereon and the roll drive assembly may include a magnetic rotor attached to the base or the casing of the instrument drive unit, and a stator attached to the other of the base or the casing of the instrument drive unit. The roll drive assembly may be configured to rotate the instrument drive unit about the longitudinal axis and relative to the base.

In aspects, the base may include a spine configured to slidably couple to the robotic arm, a proximal flange extending from a proximal end portion of the spine and rotatably supporting a proximal end portion of the instrument drive unit, and a distal flange extending from a distal end portion of the spine and rotatably supporting a distal end portion of the instrument drive unit.

In aspects, the distal flange may define a central annular opening having the distal end portion of the instrument drive unit extending therethrough.

In aspects, the magnetic rotor may be rotatably supported by the flange and encircles the central annular opening. The stator may be fixed about the distal end portion of the instrument drive unit and concentrically aligned with the magnetic rotor.

In aspects, the motor assemblies may include four motor assemblies with each motor assembly providing one of the five degrees of rotational freedom of the instrument drive unit.

In aspects, the four motor assemblies may be arranged in a circle about the longitudinal axis.

In aspects, the casing and the four motor assemblies may be configured rotate together about the longitudinal axis.

In accordance with further aspects of the disclosure, an instrument drive unit of a surgical robotic system is provided that includes a casing configured to rotate about a longitudinal axis thereof, and a cluster of electric motor assemblies supported in the casing and rotatable with the casing. The casing defines a longitudinal bore therethrough configured for routing communication cables. One of the electric motor assemblies includes a rotor rotatably supported in the casing, a stator fixed to and positioned within the casing, and an output shaft rotatably coupled to the rotor. The output shaft includes a proximal end portion non-rotatably coupled to the rotor, a distal end portion configured to interface with a corresponding driven member of a surgical instrument, and a plurality of flexure sections positioned between the proximal and distal end portions of the output shaft and configured to flex under a torque load. The instrument drive unit further includes a proximal encoder positioned proximally of the flexure sections and configured to determine a rotational position of the proximal end portion of the output shaft, and a distal encoder positioned distally of the flexure sections and configured to determine a rotational position of the distal end portion of the output shaft. A difference between the determined rotational positions corresponds with a torque of the output shaft. The instrument drive unit further includes an electronic disc assembly supported on the casing and a slip ring secured to the electronic disc assembly. The electronic disc assembly includes a plurality of printed circuit boards, each of which being in electrical communication with a respective electric motor assembly of the cluster of electric motor assemblies. The slip ring is configured to rotatably support a proximal end portion of the instrument drive unit on a surgical robotic arm.

In aspects, the instrument drive unit may further include a plurality of stator coils fixed about a distal end portion of the instrument drive unit.

In accordance with additional aspects of the disclosure, a robotic arm assembly is provided that includes a base configured to couple to a robotic arm, an instrument drive unit, and a roll drive assembly. The instrument drive unit includes a casing rotatably supported on the base, a cluster of electric motor assemblies supported in the casing and rotatable with the casing, and proximal and distal encoders. one of the electric motor assemblies includes a rotor rotatably supported in the casing, a motor stator fixed to and positioned within the casing, and an output shaft rotatably coupled to the rotor. The output shaft includes a proximal end portion non-rotatably coupled to the rotor, a distal end portion configured to interface with a corresponding driven member of a surgical instrument, and a plurality of flexure sections positioned between the proximal and distal end portions of the output shaft and configured to flex under a torque load. The proximal encoder is positioned proximally of the plurality of flexure sections and configured to determine a rotational position of the proximal end portion of the output shaft. The distal encoder is positioned distally of the plurality of flexure sections and configured to determine a rotational position of the distal end portion of the output shaft. A difference between the determined rotational positions corresponds with a torque of the output shaft. The roll drive assembly includes a rotor attached to the base or the casing of the instrument drive unit, and a stator attached to the other of the base or the casing of the instrument drive unit. The roll drive assembly is configured to rotate the instrument drive unit about a longitudinal axis of the instrument drive unit and relative to the base.

Further details and aspects of exemplary aspects of the disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the disclosure are described herein with reference to the accompanying drawings, wherein:

FIG. 1 is a schematic illustration of a surgical robotic system including a control tower, a console, and one or more surgical robotic arms;

FIG. 2 is a perspective view of a surgical robotic arm of the surgical robotic system of FIG. 1;

FIG. 3 is perspective view illustrating an instrument drive unit attached to a base of the surgical robotic arm of FIG. 2;

FIG. 4 is a perspective view, with parts separated, illustrating the instrument drive unit and the base of FIG. 3;

FIG. 5 is a perspective view, with some parts shown in phantom, of the instrument drive unit of FIG. 4;

FIG. 6 is a longitudinal cross-section of the instrument drive unit of FIG. 3 illustrating internal components thereof; and

FIG. 7 is side view illustrating a motor assembly of the instrument drive unit of FIG. 3.

### DETAILED DESCRIPTION

Embodiments of the disclosed surgical robotic system are described in detail with reference to the drawings, in which like reference numerals designate identical or corresponding elements in each of the several views. As used herein the term “distal” refers to that portion of the surgical robotic system or component thereof, that is closer to a patient, while the term “proximal” refers to that portion of the surgical robotic system or component thereof, that is further from the patient.

As will be described further herein, the disclosure provides a surgical robotic system including an instrument drive unit for driving an operation of an attached surgical instrument. The instrument drive unit includes a canister or shell and a plurality of motor assemblies arranged in a circular pattern and supported therein for mechanical coupling with driven members or shafts of the surgical instrument. Each motor assembly has a rotatable rotor rotatably supported in the canister, and a brushless motor drive stator coil embedded within the canister. The canister has a self-rotating motor magnet or stator coil to provide rotational motion to the instrument drive unit and the attached surgical instrument. A cluster of output gearheads, for example, concentric planetary gearing or off-centered spur gearing, are provided within the canister and couple with drive shafts of each rotor. An axial alignment of the output gear with the instrument drive axes is derived from the combination of each motor maximum diameter and gearhead off-center distance. An output shaft extends from each gearhead and has a flexure section that provides torsional flex under torque load in either direction.

Each instrument drive motor has a series of encoders, such as an absolute or incremental encoder at a motor rotor shaft, an absolute non-contact encoder before the flexure section, and another absolute non-contact encoder after the flexure section. The encoders provide for rotor position information for commutation, angular position, speed, and direction, and monitor the amount of flex under torque load. The difference in encoder readings before and after the flexure section of each output shaft is computed to derive torque. The canister holds all of the motor drive electronics, encoder signal processing, component identification electronics, and a localized communication bus. The canister also holds a disc type slip ring module stacked along with other printed circuit board assemblies.

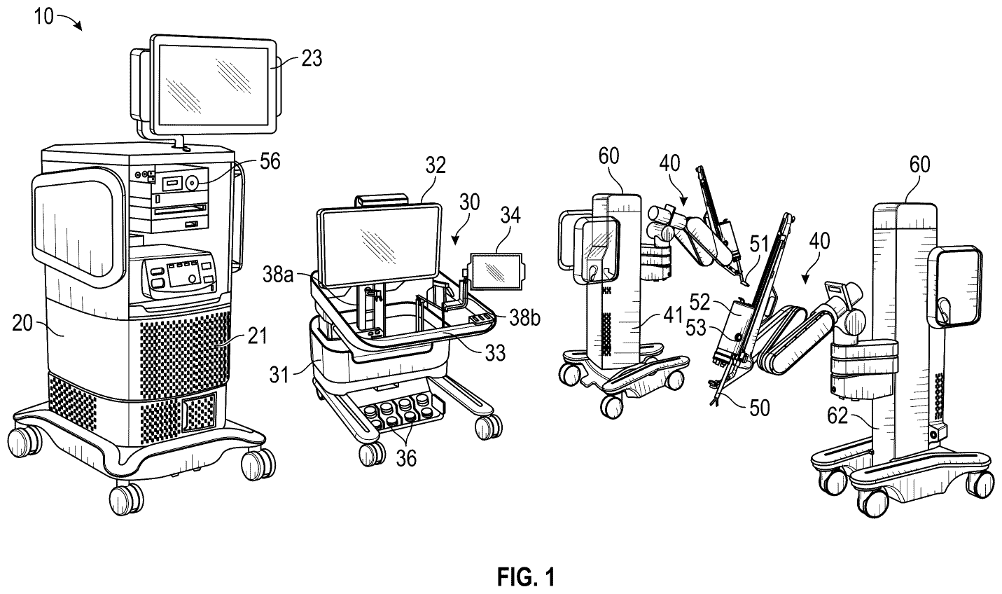

With reference to FIG. 1, a surgical robotic system 10 includes a control tower 20, which is connected to all of the components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The surgical instrument 50 is configured for use during minimally invasive surgical procedures. In embodiments, the surgical instrument 50 may be configured for open surgical procedures. In embodiments, the surgical instrument 50 may be an endoscope, such as an endoscopic camera 51, configured to provide a video feed for the user. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the disclosure and output the processed video stream.

The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

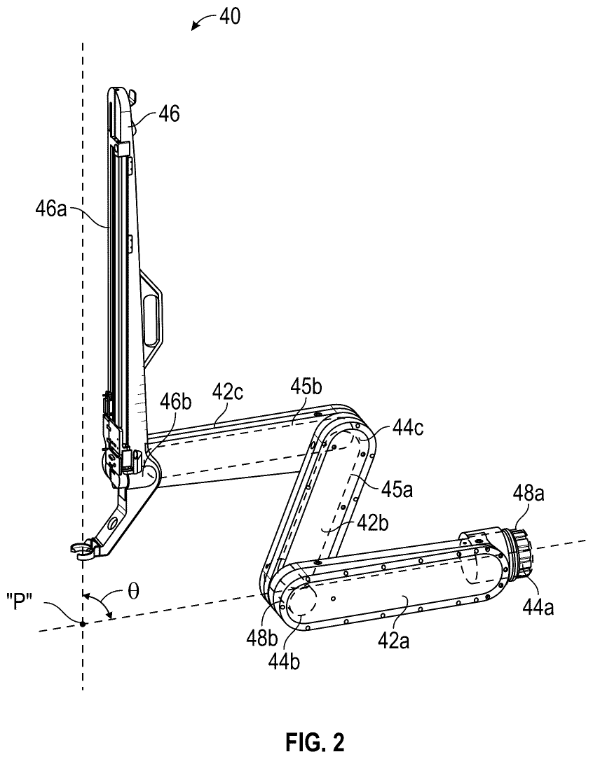

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. The robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. The robotic arm 40 also includes a plurality of manual override buttons 53 (FIG. 1) disposed on the IDU 52 and the setup arm 62, which may be used in a manual mode. The user may press one or more of the buttons 53 to move the component associated with the button 53.

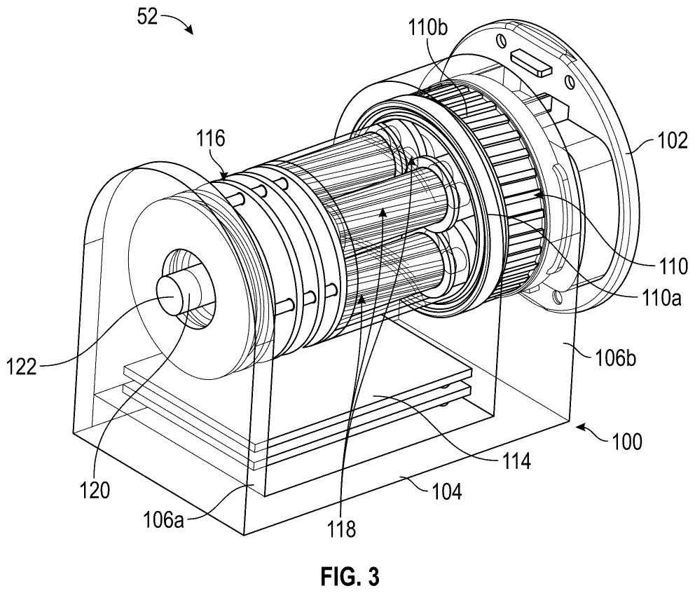

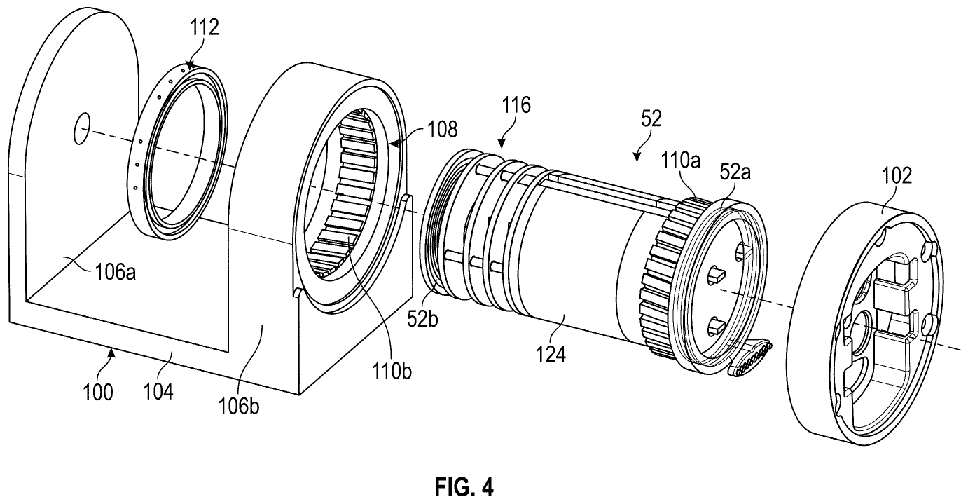

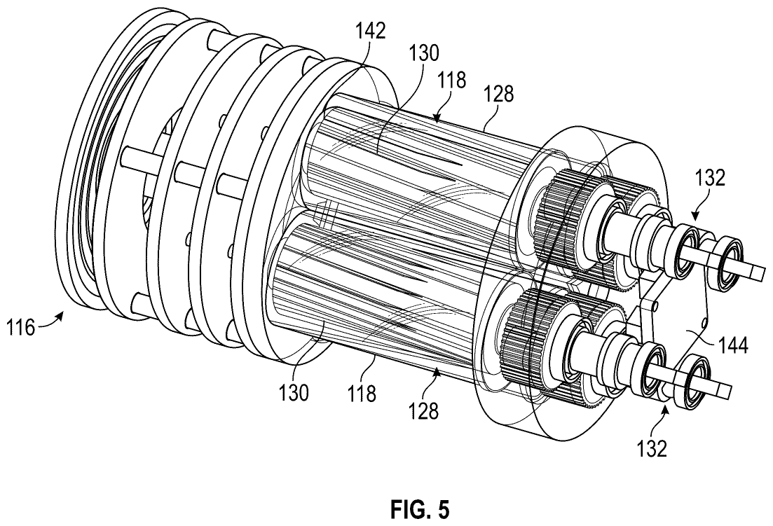

With reference to FIGS. 3-7, IDU 52 is configured to couple to a sterile interface module (not explicitly shown) that interconnects IDU 52 and surgical instrument 50. An interface 102 (FIGS. 3 and 4) may be provided that couples the IDU 52 to the sterile interface module. IDU 52 is rotatably supported on a base or slider 100 that is configured to be slidably supported on the sliding mechanism 46a (FIG. 2) of the holder 46 of the robotic arm 40.

The base 100 has a spine or support 104, a proximal flange 106a extending perpendicular from a proximal end of the spine 104, and a distal flange 106b extending perpendicular from a distal end of the spine 104. The spine 104 is supported on the holder 46, and the proximal and distal flanges 106a, 106b are spaced from one another along a length of the spine 104. The proximal flange 106a of the base 104 rotatably supports a proximal end portion 52a (FIG. 4) of the IDU 52, and the distal flange 106b of the base 100 rotatably supports a distal end portion 52b of the IDU 52. More specifically, the distal flange 106b defines an enlarged circular opening 108 through which the distal end portion 52b of the IDU 52 extends.

The IDU 52 includes a roll drive motor assembly 110 that includes a stator 110a (e.g., a plurality of stator coils) fixed about the distal end portion 52b of the IDU 52, and a roll drive rotor magnet 110b fixed within the enlarged opening 108 of the distal flange 106b. Alternately, the rotor 110b may be fixed about the IDU 52 whereas the stator 110a may be fixed to the second flange 106b of the base 100. A bearing 112 may be positioned concentrically between the rotor magnet 110b and the stator 110a.

Roll drive electronics 114 (see FIG. 3) may be attached to the spine 104 of the base 102. In other aspects, the roll drive electronics 114 may be stacked along with an electronic disc assembly 116, which are electrically connected through axial cable routing. In aspects, instead of using an AC motor assembly 110 to rotate the IDU 52, the IDU 52 may be rotated via a belt-drive or a gear mechanism of the base 100.

The electronic disc assembly 116 of the IDU 52 includes a plurality of stacked electronic circuit boards, with one board electrically connected to each motor assembly 118 of the IDU 52 and a 5^{th} axis motor board electrically connected to the stator or rotor magnet 110a, 110b of the roll drive motor assembly 110. Each of the circuit boards of the electronic disc assembly 116 may be electrically connected to a respective motor assembly 118 of the IDU 52 via electric wiring (not explicitly shown). IDU 52 may include a slip ring 120 (FIG. 3) secured to the electronic disc assembly 116 and rotatably supports the proximal end portion 52a of IDU 52 on the proximal flange 106a of the base 100. The slip ring 120 is configured to transfer power and/or data by electrical signals to/from IDU 52 and/or surgical instrument 50 (and/or the sterile interface module coupled between IDU 52 and surgical instrument 50). An encoder magnet 122 (FIG. 3) may be secured to the slip ring 120 for monitoring the rotational position and/or rotational speed of the IDU 52.

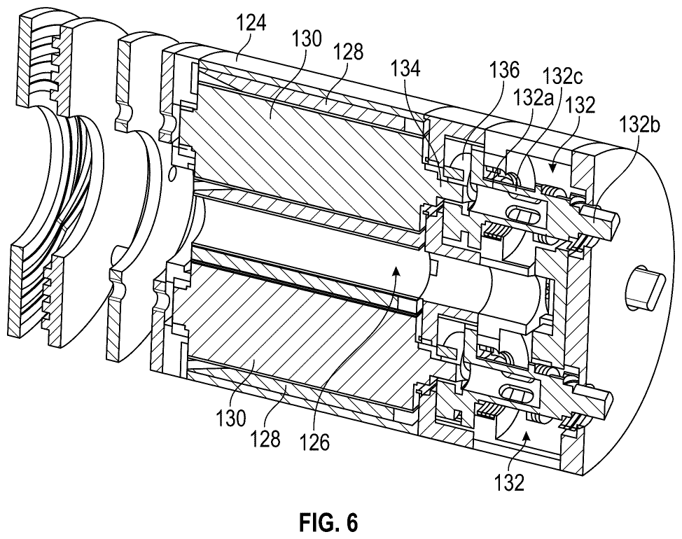

With reference to FIGS. 4-7, the IDU 52 includes a canister or casing 124 extending from and connected to the electronic disc assembly 116 and having the plurality of electric motor assemblies 118 (e.g., four brushless DC motors) supported in the casing 124. The casing 124 defines a longitudinal bore 126 (FIG. 6) therethrough configured for routing communication bus cables. The motor assemblies 118 are clustered within the casing 124 and each motor assembly 118 includes a stator 128 (e.g., a stator coil), a rotor magnet 130, and an output shaft 132. A drive shaft 134 (FIG. 6) extends from each of the rotor magnets 130 and has a gear assembly 136, such as, for example, a concentric planetary gear set or an off-centered spur gear, coupled thereto. The output shaft 132 of each motor assembly 118 has a proximal end portion 132a coupled to the gear set 136 and a distal end portion 132b configured to non-rotatable couple to a driven shaft of the surgical instrument 50. The output shaft 132 further includes a plurality of discrete flexure sections 132c positioned between the proximal and distal end portions 132a, 132b. The flexure sections 132s are configured to flex under a torsional load.

While canister or casing 124 is shown and described as including four electric motor assemblies 118, it is envisioned and within the scope of the disclosure, for canister or casing 124 to include any multiplicity of electric motors 118, for example, two, three, five, etc.

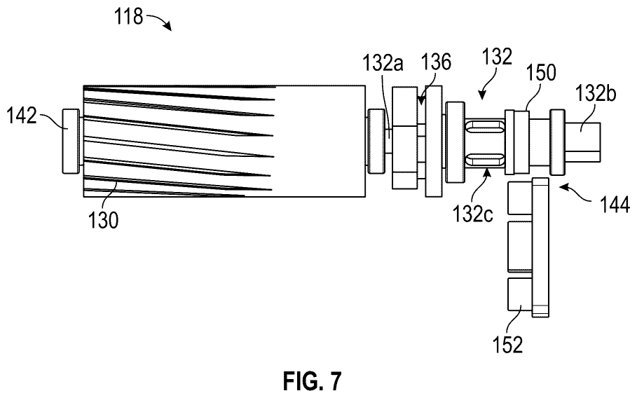

Each motor assembly 118 further includes a series of encoders including an absolute non-contact, proximal magnetic encoder 142 positioned proximally of the flexure sections 132c of the output shaft 132, and an absolute non-contact, distal magnetic encoder 144 positioned distally of the flexure sections 132c of the output shaft 132. The proximal magnetic coder 142 may be positioned adjacent a proximal end of the rotor magnet 130, and the distal magnetic encoder 144 may be positioned adjacent the distal end portion 132b of the output shaft 132. In aspects, the proximal magnetic encoder 142 may be positioned adjacent the proximal end portion 132a of the output shaft 132. As shown in FIG. 7, the distal encoder 144 may include a radial sensing magnet 150 positioned about the distal end portion 132b of the output shaft 132, and magnet sensing electronics 152 positioned adjacent the radial sensing magnet 150 for sensing a radial position of the magnet 150.

The encoders 142, 144 are configured to provide information for computation, angular position, speed, and direction, and monitors the amount of flex under torque load. The proximal magnetic encoder 142 measures the angular or rotational position of the rotor magnet 130 and the distal magnetic encoder 144 measures the angular or rotational position of the distal end portion 132b of the output shaft 132. The difference in readings (e.g., the respective rotational positions) between the proximal and distal magnetic encoders 142, 144 is computed as torque since the torque of the output shaft 132 is directly proportional to the torsional deflection at the flexure sections 132c of the output shaft 132. The measured torque may be used by one of the computers 21, 31, 41 (FIG. 1) to control an operation of the surgical instrument 50.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A robotic arm assembly comprising:
a robotic arm;
an instrument drive unit configured to provide five degrees of rotational freedom and coupled to the robotic arm, the instrument drive unit including:
a casing configured to rotate about a longitudinal axis thereof;
a cluster of electric motor assemblies supported in the casing and rotatable with the casing, at least one of the electric motor assemblies including:
a magnetic rotor rotatably supported in the casing;
a motor stator coil fixed to and positioned within the casing; and
an output shaft rotatably coupled to the magnetic rotor, the output shaft including a proximal end portion non-rotatably coupled to the magnetic rotor, a distal end portion configured to interface with a corresponding driven member of a surgical instrument, and a plurality of flexure sections positioned between the proximal and distal end portions of the output shaft and configured to flex under a torque load;
a proximal encoder positioned proximally of the plurality of flexure sections and configured to determine a rotational position of the proximal end portion of the output shaft; and
a distal encoder positioned distally of the plurality of flexure sections and configured to determine a rotational position of the distal end portion of the output shaft, wherein a difference between the determined rotational positions corresponds with a torque of the output shaft.

2. The robotic arm assembly according to claim 1, wherein the instrument drive unit includes an electronic disc assembly supported on the casing, the electronic disc assembly including a plurality of printed circuit boards, each of the printed circuit boards in electrical communication with a respective motor assembly of the plurality of motor assemblies.

3. The robotic arm assembly according to claim 1, wherein each of the motor assemblies includes a planetary gear set coupling the respective magnetic rotor with the respective output shaft.

4. The robotic arm assembly according to claim 1, further comprising:
a base coupled to the robotic arm, the base rotatably supporting the instrument drive unit thereon; and
a roll drive assembly including:
a magnetic rotor attached to the base or the casing of the instrument drive unit; and
a stator attached to the other of the base or the casing of the instrument drive unit, the roll drive assembly configured to rotate the instrument drive unit about the longitudinal axis and relative to the base.

5. The robotic arm assembly according to claim 4, wherein the base includes:
a spine configured to slidably couple to the robotic arm;
a proximal flange extending from a proximal end portion of the spine and rotatably supporting a proximal end portion of the instrument drive unit; and
a distal flange extending from a distal end portion of the spine and rotatably supporting a distal end portion of the instrument drive unit.

6. The robotic arm assembly according to claim 5, wherein the distal flange defines a central annular opening having the distal end portion of the instrument drive unit extending therethrough.

7. The robotic arm assembly according to claim 6, wherein the magnetic rotor is rotatably supported by the flange and encircles the central annular opening, the stator being fixed about the distal end portion of the instrument drive unit and concentrically aligned with the magnetic rotor.

8. The robotic arm assembly of claim 1, wherein the electric motor assemblies include four electric motor assemblies, each electric motor assembly providing one of the five degrees of rotational freedom of the instrument drive unit.

9. The robotic arm assembly of claim 8, wherein the four electric motor assemblies are arranged in a circle about the longitudinal axis.

10. The robotic arm assembly of claim 9, wherein the casing and the four electric motor assemblies are configured rotate together about the longitudinal axis.

11. An instrument drive unit of a surgical robotic system, the instrument drive unit comprising:
a casing configured to rotate about a longitudinal axis thereof, the casing defining a longitudinal bore therethrough configured for routing communication cables;
a cluster of electric motor assemblies supported in the casing and rotatable with the casing, at least one of the electric motor assemblies including:
a rotor rotatably supported in the casing;
a stator fixed to and positioned within the casing; and
an output shaft rotatably coupled to the rotor, the output shaft including a proximal end portion non-rotatably coupled to the rotor, a distal end portion configured to interface with a corresponding driven member of a surgical instrument, and a plurality of flexure sections positioned between the proximal and distal end portions of the output shaft and configured to flex under a torque load;
a proximal encoder positioned proximally of the plurality of flexure sections and configured to determine a rotational position of the proximal end portion of the output shaft;
a distal encoder positioned distally of the plurality of flexure sections and configured to determine a rotational position of the distal end portion of the output shaft, wherein a difference between the determined rotational positions corresponds with a torque of the output shaft;
an electronic disc assembly supported on the casing, the electronic disc assembly including a plurality of printed circuit boards, each of the printed circuit boards in electrical communication with a respective electric motor assembly of the cluster of electric motor assemblies; and
a slip ring secured to the electronic disc assembly and configured to rotatably support a proximal end portion of the instrument drive unit on a surgical robotic arm.

12. The instrument drive unit according to claim 11, further comprising a plurality of stator coils fixed about a distal end portion of the instrument drive unit.

13. The instrument drive unit according to claim 11, wherein each of the electric motor assemblies includes a planetary gear set coupling the respective rotor with the respective output shaft.

14. The instrument drive unit of claim 11, wherein the motor assemblies include four electric motor assemblies, each electric motor assembly providing one of the five degrees of rotational freedom of the instrument drive unit.

15. The instrument drive unit of claim 14, wherein the four motor assemblies are arranged in a circle about the longitudinal axis.

16. The instrument drive unit of claim 15, wherein the casing and the four electric motor assemblies are configured rotate together about the longitudinal axis.

17. A robotic arm assembly, comprising:
a base configured to couple to a robotic arm;
an instrument drive unit including:
a casing rotatably supported on the base;
a cluster of electric motor assemblies supported in the casing and rotatable with the casing, at least one of the electric motor assemblies including:
a rotor rotatably supported in the casing;
a motor stator fixed to and positioned within the casing; and
an output shaft rotatably coupled to the rotor, the output shaft including a proximal end portion non-rotatably coupled to the rotor, a distal end portion configured to interface with a corresponding driven member of a surgical instrument, and a plurality of flexure sections positioned between the proximal and distal end portions of the output shaft and configured to flex under a torque load;
a proximal encoder positioned proximally of the plurality of flexure sections and configured to determine a rotational position of the proximal end portion of the output shaft; and
a distal encoder positioned distally of the plurality of flexure sections and configured to determine a rotational position of the distal end portion of the output shaft, wherein a difference between the determined rotational positions corresponds with a torque of the output shaft; and
a roll drive assembly including:
a rotor attached to the base or the casing of the instrument drive unit; and
a stator attached to the other of the base or the casing of the instrument drive unit, the roll drive assembly configured to rotate the instrument drive unit about a longitudinal axis of the instrument drive unit and relative to the base.

18. The robotic arm assembly according to claim 17, wherein the instrument drive unit includes an electronic disc assembly supported on the casing, the electronic disc assembly including a plurality of printed circuit boards, each of the printed circuit boards in electrical communication with a respective motor assembly of the plurality of motor assemblies.

19. The robotic arm assembly according to claim 17, wherein each of the motor assemblies includes a planetary gear set coupling the respective rotor with the respective output shaft.

20. The robotic arm assembly according to claim 17, wherein the base includes:
a spine configured to slidably couple to the robotic arm;
a proximal flange extending from a proximal end portion of the spine and rotatably supporting a proximal end portion of the instrument drive unit; and
a distal flange extending from a distal end portion of the spine and rotatably supporting a distal end portion of the instrument drive unit.
